(19) United States
(12) Patent Application Publication
(10) Pub. No.: US 2003/0203162 A1
(43) Pub. Date: Oct. 30, 2003

(54) Methods for making nonwoven materials on a surface having surface features and nonwoven materials having surface features

(75) Inventors: Christopher Dale Fenwick, Alpharetta, GA (US); Bryan David Haynes, Cumming, GA (US); Kurtis Lee Brown, Alpharetta, GA (US); Susan Carol Paul, Alpharetta, GA (US); Christian Michael Trusock, Cumming, GA (US); Melpo Lambidonis, Cumming, GA (US); Stephen Avedis Baratian, Atlanta, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/136,702

(22) Filed: Apr. 30, 2002

(51) Int. Cl. B32B 3/00; B32B 3/28; B32B 3/30; B32B 1/00; B32B 27/12; B65C 1/00; A61L 2/00; B05D 3/00; B29D 1/00; D06C 3/00; B05D 3/12; D06C 5/00; B21F 9/00; B05D 7/22; B05D 5/00

(52) U.S. Cl. 428/156; 428/174; 428/179; 442/394; 156/214; 427/2.31; 427/171; 427/230; 427/243

(57) ABSTRACT

A process of making a nonwoven fabric comprising providing a three-dimensional surface that comprises surface features that are air permeable, depositing fibers or a web comprising fibers onto the surface, and stabilizing the fibers to form a nonwoven fabric is provided.

### CROSS-REFERENCE TO RELATED APPLICATIONS

This application is related to commonly assigned U.S. patent application Ser. No. ______, entitled “NONWOVEN MATERIALS HAVING SURFACE FEATURES” filed by Express Mail Procedure EL 471213693 US contemporaneously herewith and which is hereby incorporated by reference herein.

### FIELD

The present invention is directed to nonwoven materials and methods of making nonwoven materials.

### BACKGROUND

Nonwoven fabrics are useful for a wide variety of applications, including absorbent personal care products, garments, medical products, and cleaning products. Nonwoven personal care products include infant care items such as diapers, child care items such as training pants, feminine care items such as sanitary napkins, and adult care items such as incontinence products. Nonwoven garments include protective workwear and medical apparel such as surgical gowns. Other nonwoven medical products include nonwoven wound dressings and surgical dressings. Cleaning products that contain nonwovens include towels and wipes. Still other uses of nonwoven fabrics are well known. The foregoing list is not considered exhaustive.

Various properties of nonwoven fabrics determine the suitability of nonwoven fabrics for different applications. Nonwoven fabrics may be engineered to have different combinations of properties to suit different needs. Variable properties of nonwoven fabrics include liquid-handling properties such as wettability, distribution, and absorbency, strength properties such as tensile strength and tear strength, softness properties, durability properties such as abrasion resistance, and aesthetic properties. The physical shape of a nonwoven fabric also affects the functionality and aesthetic properties of the nonwoven fabric. Nonwoven fabrics are initially made into sheets which, when laid on a flat surface, may have a substantially planar, featureless surface or may have an array of surface features such as apertures or projections, or both. Nonwoven fabrics with apertures or projections are often referred to as three-dimensional or shaped nonwoven fabrics. The present invention relates to three-dimensional or shaped nonwoven fabrics.

The manufacture of nonwoven fabrics is a highly developed art. Generally, nonwoven webs and their manufacture involve forming filaments or fibers and depositing the filaments or fibers on a carrier in such a manner so as to cause the filaments or fibers to overlap or entangle. Depending on the degree of web integrity desired, the filaments or fibers of the web may then be bonded by means such as an adhesive, the application of heat or pressure, or both, sonic bonding techniques, or entangling by needles or water jets, and so forth. There are several methods of producing fibers or filaments within this general description; however, two commonly used processes are known as spunbonding and meltblowing and the resulting nonwoven fabrics are known as spunbond and meltblown fabrics, respectively.

Generally described, the process for making spunbond nonwoven fabrics includes extruding thermoplastic material through a spinneret, quenching and drawing the extruded material into filaments with a stream of high-velocity air to form a random web on a forming surface. Such a method is referred to as meltspinning. Spunbond processes are generally defined in numerous patents including, for example, U.S. Pat. No. 3,802,817 to Matsuki et al.; U.S. Pat. No. 4,692,618 to Dorschner, et al.; U.S. Pat. No. 4,340,563 to Appel, et al.; U.S. Pat. Nos. 3,338,992 and 3,341,394 to Kinney; U.S. Pat. No. 3,502,538 to Levy; U.S. Pat. Nos. 3,502,763 and 3,909,009 to Hartmann; U.S. Pat. No. 3,542,615 to Dobo, et al.; and Canadian Patent No. 803,714 to Harmon.

On the other hand, meltblown nonwoven fabrics are made by extruding a thermoplastic material through one or more dies, blowing a high-velocity stream of air, usually heated air, past the extrusion dies to generate an air-conveyed meltblown fiber curtain and depositing the curtain of fibers onto a forming surface to form a random nonwoven web. Meltblowing processes are generally described in numerous publications including, for example, an article titled “Superfine Thermoplastic Fibers” by Wendt in Industrial and Engineering Chemistry, Vol. 48, No. 8, (1956), at pp. 1342-1346, which describes work done at the Naval Research Laboratory in Washington, D.C.; Naval Research Laboratory Report 111437, dated Apr. 15, 1954; U.S. Pat. Nos. 4,041,203, 3,715,251, 3,704,198, 3,676,242 and 3,595,245; and British Specification 1,217,892.

Spunbond and meltblown nonwoven fabrics can usually be distinguished by the diameters and the molecular orientation of the filaments or fibers which form the fabrics. The diameter of spunbond and meltblown filaments or fibers is the average cross-sectional dimension. Spunbond filaments or fibers typically have average diameters greater than 6 microns and often have average diameters in the range of 12 to 40 microns. Meltblown fibers typically have average diameters of less than 6 microns. However, because larger meltblown fibers, having diameters of at least 6 microns may also be produced, molecular orientation can be used to distinguish spunbond and meltblown filaments and fibers of similar diameters. For a given fiber or filament size and polymer, the molecular orientation of a spunbond fiber or filament is typically greater than the molecular orientation of a meltblown fiber. Relative molecular orientation of polymeric fibers or filament can be determined by measuring the tensile strength and birefringence of fibers or filaments having the same diameter.

Tensile strength of fibers and filaments is a measure of the stress required to stretch the fiber or filament until the fiber or filament breaks. Birefringence numbers are calculated according to the method described in the spring 1991 issue of INDA Journal of Nonwovens Research, (Vol. 3, No. 2, p. 27). The tensile strength and birefringence numbers of polymeric fibers and filaments vary depending on the particular polymer and other factors; however, for a given fiber or filament size and polymer, the tensile strength of a spunbond fiber or filament is typically greater than the tensile strength of a meltblown fiber and the birefringence number of a spunbond fiber or filament is typically greater than the birefringence number of a meltblown fiber.

A number of patents describe methods for making shaped or three-dimensional nonwoven fabrics: for example, U.S. Pat. Nos. 5,575,874 and 5,643,653 issued to Griesbach et al.; U.S. Pat. No. 4,741,941 issued to Engelbert et al.; and U.S. Pat. Nos. 6,331,268, 6,331,345 and 6,455,319 issued to Kauschke et al. Despite prior advances in the art, there is still a need for improved nonwoven fabrics having surface features and methods for forming such nonwoven fabrics.

### SUMMARY

In response to the difficulties and problems encountered in the prior art, new nonwoven materials and methods of making nonwoven materials have been discovered. The present invention provides a nonwoven material that includes surface features and methods for making nonwovens. In accordance with the present invention a method of making a nonwoven fabric is described. The method includes providing a surface that has surface features that are air permeable and includes depositing fibers onto the surface. Desirably, the surface features have an air permeability that is substantially equal to the air permeability of the portion or portions of the surface that do not include surface features.

In one embodiment, the surface includes a plurality of surface features at least one of which is greater than ⅛ of an inch in height. In another embodiment, the surface includes a plurality of surface features at least one of which is greater than 5/32. In yet another embodiment, the surface includes a plurality of surface features at least one of which is greater than 3/16 of an inch in height. In still yet another embodiment, the surface includes a plurality of surface features at least one of which is greater than ¼ of an inch.

In yet another desirable embodiment, the surface is a permeable metal surface, for example a metal screen that includes three-dimensional surface features such as projections or depressions. Desirably, the surface, including the surface features, is substantially uniformly permeable over the majority of the surface. In one embodiment, the fibers are bicomponent fibers. In another embodiment, the fibers are bicomponent fibers having a side-by side or a sheath/core configuration. The methods of the present invention may further include bonding the fibers at elevated temperature. In an illustrated embodiment, the method for making a nonwoven fabric includes the steps of: providing polymeric filaments; forming the polymeric filaments on a surface that includes surface features, wherein the surface and the surface features are air permeable; forcing air or another gas through the polymeric filaments, the surface and the surface features to arrange the polymeric filaments into a web; and bonding the polymeric filaments to integrate the web. In a desirable embodiment of the illustrated embodiments, a plurality of the surface features have a height that is greater than ⅛ of an inch, more desirably a plurality of the surface features are macroscopic and have a height that is greater than 3/16 of an inch and even more desirably greater than ¼ of an inch. A plurality of the surface features may also have a basal dimension that is greater than ⅛ of an inch.

Methods of the present invention may include a step of forming polymeric filaments. For example, a method of the present invention may include a step of forming filaments by melt spinning and then depositing the filaments on a surface having surface features. Alternatively, the nonwoven web may be a carded web that is first formed and then deposited on a surface where the carded web is then contacted with heated, forced air to conform the carded web to the surface and bond the fibers of the web. Nonwoven webs of the present invention may also include cellulose fibers. Methods of the present invention may include a step of drawing the polymeric filaments, and, may further include a step of quenching the filaments. Once the fibers of the nonwoven web are bonded and the shape of the web is set, the web may be separated from the surface.

The present invention also includes nonwoven fabrics made by method of present invention. The nonwoven materials of the present invention may be used in absorbent products with the absorbent portion or layer of the absorbent product placed adjacent the bottom surface of the nonwoven material. For example, the nonwoven material of the present invention can be used as a body-side liner of a diaper.

### BRIEF DESCRIPTION OF THE DRAWINGS

FIG. 1 is a schematic illustration of one method of making a nonwoven web with surface features.

FIG. 2 schematically illustrates a wire having surface features.

FIG. 3 schematically illustrates a nonwoven web including surface features.

FIGS. 4A and 4B are photographs of two nonwoven webs including surface features that were made by methods described herein.

### DEFINITIONS

As used herein the following terms have the specified meanings, unless the context demands a different meaning, or a different meaning is expressed; also, the singular generally includes the plural, and the plural generally includes the singular unless otherwise indicated.

Words of degree, such as “about”, “substantially”, and the like are used herein in the sense of “at, or nearly at, when given the manufacturing and material tolerances inherent in the stated circumstances” and are used to prevent the unscrupulous infringer from unfairly taking advantage of the invention disclosure where exact or absolute figures are stated as an aid to understanding the invention.

As used herein, the term “absorbent product” or “personal care absorbent product” means diapers, training pants, swim wear, absorbent underpants, adult incontinence products, sanitary wipes, wipes, feminine hygiene products, wound dressings, nursing pads, time release patches, bandages, mortuary products, veterinary products, hygiene and so forth.

As used herein, the terms “comprises”, “comprising” and other derivatives from the root term “comprise” are intended to be open-ended terms that specify the presence of any stated features, elements, integers, steps, or components, but do not preclude the presence or addition of one or more other features, elements, integers, steps, components, or groups thereof.

As used herein, the term “fabric” refers to all of the woven, knitted and nonwoven fibrous webs.

As used herein, the term “fiber” refers to a threadlike object or structure from which textiles and nonwoven fabrics are commonly made. The term “fiber” is meant to encompass both continuous and discontinuous filaments, and other threadlike structures having a length that is substantially greater than that its diameter.

As used herein, the term “macroscopic surface features” are three-dimensional features that extend from the surface and are large enough to be perceived or examined with the unaided eye, desirably such features have at least one dimension that is greater than ⅛ of an inch (˜3 mm), more desirably greater than 5/32 of an inch (˜4 mm), more desirably greater than 3/16 of an inch (˜5 mm) and even more desirably such features have at least one dimension greater than one quarter of an inch (˜6 mm).

As used herein the term “meltblown fibers” means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a forming surface to form a web of randomly dispersed meltblown fibers. Such a process is disclosed, for example, in U.S. Pat. No. 3,849,241 to Butin et al. Meltblown fibers are microfibers, which may be continuous or discontinuous, are generally smaller than 10 microns in average diameter, and are generally tacky when deposited onto a forming surface.

As used herein “multilayer laminate” means a laminate including two or more layers of material laminated into a finished structure. For example, one or more of the layers may be a spunbond layer and/or some of the layers may be a meltblown layer. One specific example of a multilayer laminate is a spunbond/meltblown/spunbond (SMS) laminate. Other multilayer laminates are disclosed in U.S. Pat. No. 4,041,203 to Brock et al., U.S. Pat. No. 5,169,706 to Collier, et al, U.S. Pat. No. 5,145,727 to Potts et al., U.S. Pat. No. 5,178,931 to Perkins et al. and U.S. Pat. No. 5,188,885 to Timmons et al. A multilayer laminate may be made by sequentially depositing onto a moving forming belt first a spunbond fabric layer, then a meltblown fabric layer and last another spunbond layer and then bonding the laminate in a manner described below. Alternatively, the fabric layers may be made individually, collected in rolls, and combined in a separate bonding step. Such fabrics usually have a basis weight of from about 0.1 to 12 ounces per square yard (3 to 400 grams per square meter), or more particularly from about 0.75 osy to about 3 osy. Multilayer laminates may also have various numbers of meltblown layers or multiple spunbond layers in many different configurations and may include other materials like films (F) or coform materials, e.g. SMMS, SM, SFS, and so forth.

As used herein the terms “nonwoven” and “nonwoven fabric or web” mean a web having a structure of individual fibers, filaments or threads which are interlaid, but not in an identifiable manner as in a knitted fabric. Nonwoven fabrics or webs have been formed from many processes such as for example, meltblowing processes, spunbonding processes, and bonded carded web processes. The basis weight of nonwoven fabrics is usually expressed in ounces of material per square yard (osy) or grams per square meter (gsm) and the fiber diameters useful are usually expressed in microns. (Note that to convert from osy to gsm, multiply osy by 33.91).

As used herein the term “spunbonded webs” refers to webs comprising small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al. Spunbond fibers are generally not tacky when they are deposited onto a forming surface. Spunbond fibers are generally continuous and have average diameters (from a sample of at least 10) larger than 7 microns, more often, between about 10 and 20 microns.

These terms may be defined with additional language in the remaining portions of the specification.

### DETAILED DESCRIPTION

As discussed above, the present invention provides nonwoven fabrics having surface features and methods of making nonwoven fabrics on a surface having surface features. The nonwoven fabric of the present invention can be formed by methods not requiring hydroentangling and can be used to form webs having features that assume a shape that corresponds to the shape of the surface having surface features. Advantageously, methods of the invention can be used to manufacture nonwoven fabrics having features such as projections that correspond to the features of a forming surface. These features may include apertures and/or depressions as well as projections. The present invention comprehends a relatively efficient and economical process for making such nonwoven fabrics and includes fabrics with macroscopic surface features and articles incorporating these fabrics. Nonwoven fabrics of the present invention are particularly useful for making personal care articles, garments, medical products, cleaning products, construction materials such as soundproofing and insulating materials, air filters and other filtration materials, and so forth.

In at least one illustrated embodiment, the present invention provides a method of making a nonwoven fabric that includes forming a nonwoven fabric on a surface that comprises surface features. It is particularly desirable that the surface features on the surface are air permeable. It is even more desirable that surface features of the surface have similar permeability as the non-surface regions or land areas of the surface. In a desirable embodiment, the surface features on the nonwoven fabric are macroscopic in size and provide separation between the majority of the nonwoven fabric surface and a body part that is in contact with the nonwoven. Such a nonwoven fabric is particularly useful as a body side liner in a personal care article, such as a diaper, pantiliner and so forth. It is desirable that the surface upon which the nonwoven fabric is formed includes one or more surface features that have at least one dimension, for example a height, a depth, a length or a width, that is greater than 3/64, 1/16, 3/32, ⅛, 5/32, 3/16, and even ¼ of an inch. More specifically, it is desirable that the surface upon which the nonwoven fabric is formed includes one or more surface features that are at least ⅛ of an inch, more desirably at least 5/32 of an inch, still more desirably at least 3/16, and even ¼ of an inch in height.

In an exemplary embodiment, the present invention provides a method of making a nonwoven fabric that includes depositing melt spun, continuous multicomponent polymeric fibers onto a first surface and transferring the fibers to a second surface that includes macroscopic surface features. More particularly, this embodiment includes depositing continuous bicomponent filaments that include a primary polymeric component A and a lower melting polymeric component B. It is desirable that adhesive polymeric component B melts at a lower temperature than the primary component and/or other components and acts as an adhesive to bind the fibers when the fibers are heated and then cooled. The bicomponent filaments have a cross-section, a length, and a peripheral surface. The components A and B are arranged in substantially distinct zones across the cross-section of the bicomponent filaments and extend continuously along the length of the bicomponent filaments. The adhesive component B constitutes at least a portion of the peripheral surface of the bicomponent filaments continuously along the length of the bicomponent filaments. The bicomponent spunbond filaments have an average diameter from about 6 to about 40 microns, and desirably from about 15 to about 40 microns. The components A and B can be arranged in either a side-by-side or eccentric sheath/core arrangement as to obtain filaments which exhibit crimp. Alternatively, the components A and B can be arranged in a concentric sheath core arrangement if little or no crimp is desirable. Desirably, primary polymeric component A is the core of the filament and adhesive polymeric component B is the sheath in the sheath/core arrangement. Bicomponent fibers and melt spinning are known and are described in U.S. Pat. Nos. 5,575,874 and 5,643,653 issued to Griesbach et al. which are herein incorporated by reference in their entirety. Multicomponent meltspun nonwoven fabrics and methods of making multicomponent meltspun nonwoven fabrics are also known and are described in U.S. Pat. No. 5,382,400 issued to Pike et al. which is also herein incorporated by reference in its entirety.

Methods for extruding multicomponent polymeric filaments into such arrangements are also known. A wide variety of polymers are suitable to practice the present invention including polyolefins (such as polyethylene, polypropylene and polybutylene), polyesters, polyamides, polyurethanes, and so forth. Primary component A and adhesive component B can be selected so that the resulting bicomponent filament is capable of developing a crimp or, alternatively, the fibers can be crimped mechanically. Desirably, primary polymer component A has a melting temperature which is greater than the melting temperature of adhesive polymer component B. Desirably, primary polymer component A comprises polypropylene or a random copolymer of propylene and ethylene and adhesive polymer component B comprises polyethylene or a random copolymer of propylene and ethylene. Desirable polyethylenes include linear low density polyethylene (LLDPE) and high density polyethylene (HDPE). In addition, adhesive polymer component B may comprise additives for enhancing the natural crimp of the filaments, lowering the bonding temperature of the filaments, and enhancing the abrasion resistance, strength and softness of the resulting fabric.

Suitable materials for preparing the multicomponent filaments of the fabric of the present invention include PD-3445 polypropylene available from Exxon Mobil of Houston, Tex.; a linear low density polyethylene available under the designation ASPUN 6811A, 2553 LLDPE and 61800 polyethylene available from Dow Chemical Company of Midland, Mich.; and 25355 and 12350 HDPEs available from Dow Chemical Company. When a polypropylene is component A and a polyethylene is component B, the bicomponent filaments may comprise from about 20 to about 80 percent by weight of a polypropylene and from about 80 to about 20 percent polyethylene. More desirably, the filaments may comprise from about 40 to about 60 percent by weight polypropylene and from about 60 to about 40 percent by weight polyethylene.

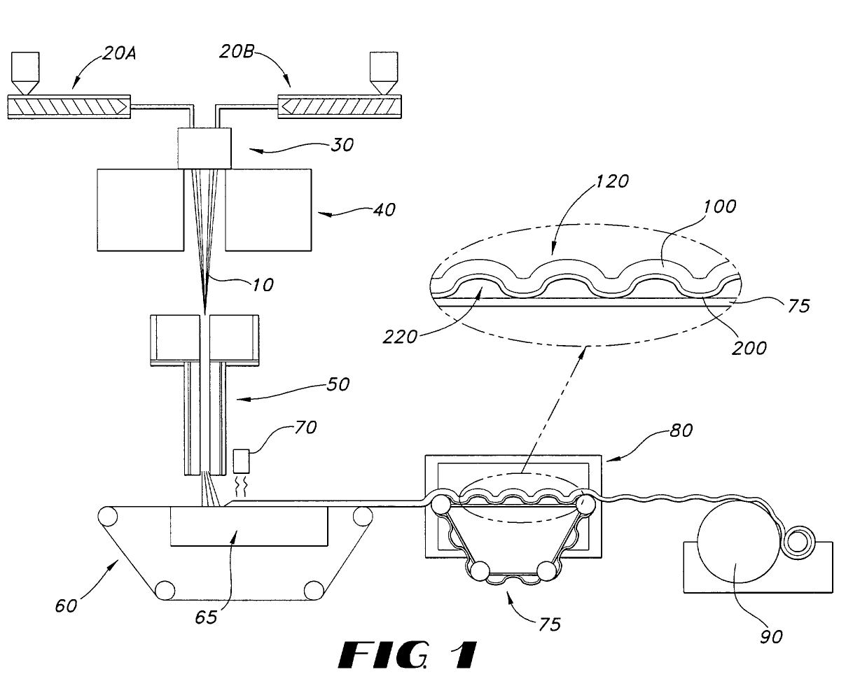

Turning to FIG. 1, an exemplary method of the present invention is disclosed. FIG. 1 illustrates a process line that is arranged to produce bicomponent continuous filaments, but it should be understood that the present invention comprehends nonwoven fabrics made with single component filaments, mixtures of filaments including cellulose-based filaments, and/or multicomponent filaments having more than two components. For example, a nonwoven fabric of the present invention can be made from filaments including pulp fibers and/or filaments having three or four or more components. The process line includes two extruders 20A and 20B. First extruder 20A extrudes the primary polymer component A and a second separate extruder 20B for extrudes the adhesive polymer component B. Polymer component A is fed into the respective extruder from a first hopper and polymer component B is fed into the respective extruder from a second hopper. Polymer components A and B are fed from the extruders 20A and 20B through respective polymer conduits to a spinneret 30. Spinnerets for extruding bicomponent filaments are known to those skilled in the art and thus are not described here in detail.

Generally described, the spinneret 30 includes a housing containing a spin pack which includes a plurality of plates stacked one on top of the other with a pattern of openings arranged to create flow paths for directing polymer components A and B separately through the spinneret 30. The spinneret 30 has openings arranged in one or more rows. The spinneret openings form a downwardly extending curtain of filaments 10 when the polymers are extruded through the spinneret 30. For the purposes of the present invention, spinneret 30 may be arranged to form side-by-side or sheath/core bicomponent filaments or other types of filaments. The process line also includes a quench air blower 40 positioned adjacent the curtain of filaments extending from the spinneret 30. Air from the quench blower 40 quenches the filaments extending from the spinneret 30. The quench air can be directed from one side of the filament curtain or both sides of the filament curtain as illustrated.

A fiber draw unit (FDU) or aspirator 50 is positioned below the quench air blower 40 and receives the quenched filaments. Fiber draw units or aspirators for use in melt spinning polymers are also known. Suitable fiber draw units for use in the process of the present invention include a linear fiber aspirator of the type described and illustrated in U.S. Pat. No. 3,802,817, linear draw system of the type described and illustrated in U.S. Pat. No. 4,340,563 and eductive guns of the type described and illustrated in U.S. Pat. Nos. 3,692,618 and 3,423,266, all of which are incorporated herein by reference. Generally, the fiber draw unit 50 includes an elongate vertical passage through which filaments are drawn by aspirating air entering from the sides of the passage and flowing downwardly through the passage.

A shaped, endless, and at least partially foraminous, forming surface 60 is positioned below the fiber draw unit 50 to collect and receive continuous filaments from the outlet opening of the fiber draw unit. The forming surface 60 may be a belt that travels around guide rollers as illustrated to provide a continuous process. Desirably, a vacuum 65 is positioned below the forming surface 60 where the filaments are deposited to draw the filaments against the forming surface 60. Although the forming surface 60 is illustrated as a belt in FIG. 1, it is understood that the forming surface can also be in other forms, for example a drum. Details of particular shaped forming surfaces are explained in more detail below.

In the embodiment illustrated in FIG. 1, the filaments that have been collected on a forming surface are exposed to a hot-air knife (HAK) 70 that provides some integrity to the web so that the web can be transferred to another wire. Transfer of a web can be accomplished without the use of a HAK and by other methods including but not limited to, vacuum transfer, compaction or compression rolls and other mechanical means. The web is then transferred to a second surface 200, for example a bonding wire (shown enlarged) that includes a surface having macroscopic surface features. In this illustrated embodiment, the second, bonding surface is located on and attached to a carrying wire that in the Examples is a conventional bonding wire 75. However, the surface having topographical features may be the carrying wire as long as the wire includes three-dimensional surface features. Furthermore, although the bonding surface 75 is illustrated as a conventional forming wire, either or both the bonding wire surface or the bonding wire and the forming surface or forming wire may include surface features. Furthermore, the wire may be continuous and one surface or wire may serve both functions. The forming surface or forming wire and/or the bonding surface or bonding wire may for example include a metal or plastic wire, mesh or screen that is manufactured to include surface features, or a drum that includes surface features and so forth as long as the surface is air permeable and includes surface features that are air permeable. Air permeability of the surface features allows fibers to collect on and conform to the surface and surface features when air is forced through the surface.

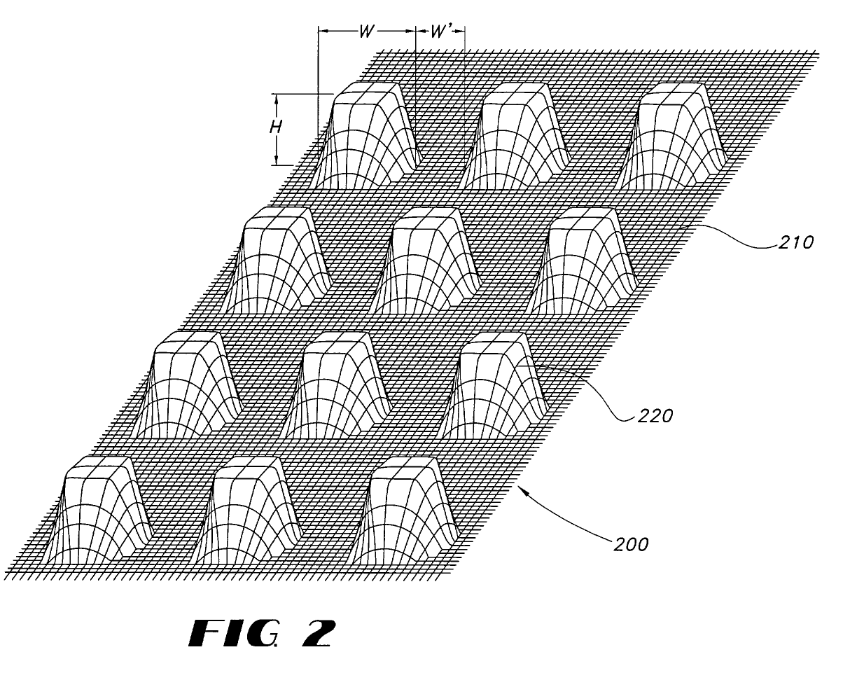

It is desirable that the surface and the surface features are uniformly permeable and exhibit a uniform pressure drop in the z-direction over the entire surface during the web making and forming process. Such a surface is illustrated in FIG. 2 and may include a forming wire that has been deformed into a three-dimensional, shaped surface.

Desirably a highly shaped surface including features that extend out of the main plane of the surface by ⅛, 5/32, 3/16, or even ¼ of an inch and have similarly sized base dimensions. The shaped surface will impart topography to a nonwoven web that is placed over this surface and then bonded. The surface includes topographical features that are not generated by blocking off zones where there is a pressure gradient over the surface of the wire, nor is the topography generated only by having material projections that have different pressure drops than the base wire. It should be understood that not all of the surface features must be air permeable. For example, some of the surface features may be impermeable to provide alternate features penetrations in a nonwoven fabric that is formed on the surface.

The process line may include one or more bonding devices such as a through-air bonder (TAB) 80. Through-air bonders are known and are therefore not disclosed here in detail. Generally, the through-air bonder 80 directs hot air through one or more nozzles against the filament web on the surface 200 and the support wire 75 below. Hot air from the nozzle of the through-air bonder 80 flows through the web and the forming surface and bonds the filaments of the web together to consolidate and form an integrated web. Alternatively or in addition, a more conventional through-air bonder that includes a perforated roller may be included in the methods of the present invention. Lastly, the process line includes a winding roll 90 for taking up the nonwoven fabric.

To operate the illustrated process line, the hoppers of extruders 20A and 20B are filled with the respective polymer components A and B. Polymer components A and B are melted and extruded by the respective extruders through polymer conduits and the spinneret 30. Although the temperatures of the molten polymers vary depending on the polymers used, when polypropylene and polyethylene are used as primary component A and adhesive component B respectively, the desirable temperatures of the polymers range from about 370° F. to about 530° F. and desirably range from 400° F. to about 450° F. As the extruded filaments 10 extend below the spinneret 30, a stream of air from the quench blower 40 at least partially quenches the filaments and may be used to develop a latent crimp in the filament if desired. Desirably, the quench air flows in a direction substantially perpendicular to the length of the filaments at a temperature of from about 45° F. to about 90° F. and at a velocity from about 100 feet per minute to about 400 feet per minute. The filaments should be quenched sufficiently before being collected on the forming surface 60 so that the filaments can be arranged by forced air passing through the filaments and the forming surface. Quenching the filaments reduces the tackiness of the filaments so that the filaments do not adhere to one another too tightly before being bonded and can be moved or arranged on a forming surface during collection of the filaments on the forming surface and formation of the web. After quenching, the filaments are drawn into the vertical passage of the fiber draw unit 50 by a flow of air through the fiber draw unit. The fiber draw unit is desirably positioned 30 to 60 inches below the bottom of the spinneret 30.

It is desirable that the filaments of the nonwoven web are crimped to provide a lofty nonwoven that conforms well to the forming surface and the surface features. And although the illustrated method of carrying out the present invention includes multicomponent filaments that are crimped, the present invention encompasses uncrimped fibers as well as fibers that are crimped by other methods, for example mechanically crimping fibers. Crimped fibers and methods of crimping fibers are known in the art. Multicomponent filaments may be contacted with heated air after quenching and upstream of the aspirator. In addition, multicomponent filaments may be contacted with heated air between the aspirator and the web-forming surface. In addition, multicomponent filaments may be contacted with heated air between after web formation on the web forming surface. Furthermore, the filaments may be heated by methods other than heated air such as exposing the filaments to electromagnetic energy such as microwaves or infrared radiation. The present invention also contemplates use of nonwoven webs made by other methods for example, bonded carded webs, meltblown webs and webs made from uncrimped filaments and/or single component filaments. For example, a carded web can be formed and deposited on a surface having surface features and then conformed to the surface features with the use of forced air at elevated temperature to conform and bond the carded web into a bonded carded web having surface features.

In the embodiment illustrated in FIG. 1 and described in Example 1 below, filaments were formed through the outlet opening of the fiber draw unit 50 and then deposited onto a traveling forming surface 60. As the filaments 10 contact the forming surface 60, a vacuum 65 box draws the filaments against the forming surface to form an unbonded, nonwoven web of continuous filaments. In Example 2, the forming surface included a surface having macroscopic surface features 220 and the filaments assumed a shape corresponding to the shape of the surface 200. Because the filaments are quenched, the filaments are not too tacky and the vacuum can move or arrange the filaments on the surface and the surface features as the filaments are being collected on the surface and formed into a web. If the filaments are too tacky, the filaments stick to one another and cannot be arranged on the surface during formation of the nonwoven web.

In Example 2 below, after the filaments were collected on a forming surface having surface features, the filaments were conveyed to a TAB 80 with the forming surface 200 for bonding. In Example 1 below, the unbonded web of filaments was transferred to a second support wire 75 having a surface 200 including surface features 220 and attached to the support wire. The support wire, surface having features and nonwoven web were then conveyed to the TAB. In the TAB, the filaments were bonded by the elevated temperatures under high air pressure while the web was still on the forming surface 200 SO that the web conformed to and retained the shape imparted by the forming surface. The nonwoven webs formed in these examples included surface features that retained their shapes when removed from the forming surface.

The TAB directs a flow of air having a temperature above the melting temperature of the adhesive component B through the web and forming surface. It is particularly desirable that the forming surface and the surface features on the forming surface are permeable to air. It is particularly desirable that surface features on the forming surface and the portion or portions of the forming surface that do not include surface features have equal or similar air permeabilities. Desirably, the hot air contacts the web across the entire width of the web. The hot air melts the lower melting adhesive component B and thereby forms bonds between the bicomponent filaments to integrate the web. When polypropylene and polyethylene are used as polymer components A and B respectively, the air flowing from the TAB desirably has a temperature at the web surface ranging from about 230° F. to about 500° F. and a velocity at the web surface from about 100 feet per minute to about 5000 feet per minute. However, the temperature and the velocity of the air from TAB may vary depending on factors such as the polymers which form the filaments, the thickness of the web, the area of web surface contacted by the air flow, and the line speed of the forming surface. After being bonded within the TAB, the fabric may be transferred from the forming surface 200 to a winding roll 90 and collected or, alternatively, directed for further processing or treatment.

When used to make liquid absorbent articles, a nonwoven fabric made by a method of the present invention may be treated with conventional surface treatments or contain conventional polymer additives to enhance the wettability of the fabric. For example, the nonwoven fabric may be treated with polyalkylene-oxide modified siloxanes and silanes such as polyalkylene-oxide modified polydimethyl-siloxane as disclosed in U.S. Pat. No. 5,057,361. Such a surface treatment enhances the wettability of the fabric. The nonwoven web may be treated before it is wound onto the winding roller 90. The nonwoven web is then ready for further treatment or use.

When the spunbond filaments are crimped, the fabric of the present invention advantageously results in a relatively high loft material that is also relatively resilient. The crimp of the filaments creates an open web structure with substantial void portions between filaments and the filaments are bonded at points of contact of the filaments. Again, although the nonwoven fabric described above is made with bicomponent filaments, it should be understood that nonwoven fabrics of the present invention may be made with single component spunbond filaments, meltblown filaments, bonded carded webs, air laid webs and so forth. For example, single component spunbond filaments can be made in the same manner as described above with regard to FIG. 1 and Examples 1 and 2 except that the spinneret will be adapted to make single component filaments. See, for example, the patents previously identified with respect to spunbond processes. Furthermore, the fibers may be bonded by adding the adhesive polymeric component in another manner. An SMS multilayer laminate having surface features can be formed on a surface having such features and forcing hot air through the SMS and the permeable surface having surface features that the SMS is placed on. Alternatively, if the nonwoven fabric is deposited or otherwise formed onto a surface having features using forced air, the nonwoven fabric can be bonded in a separate step of applying heat that does not necessarily use forced air.

One method of making a fabric of the present invention with single component filaments is to combine a polymeric bonder powder with the filaments during collection of the filaments on the forming surface or deposition of the filaments on the forming surface and bond the filaments while the web is still on the forming surface. Another suitable method of making a fabric of the present invention with single component filaments is to simultaneously spin spunbond adhesive filaments or melt meltblown filaments with the primary single component filaments. Yet another method is to combine single component staple length adhesive fibers with the primary filaments during collection of the primary filaments on the forming surface or deposition on the forming surface. Generally, it is desirable to stabilize the web structure while the web is in contact with the surface having surface features in order to set or otherwise stabilize topography in the web. With any of these methods, the web may be bonded in the same manner as the multicomponent filaments are bonded. The web can be stabilized for example, by heating the web, or by other means, for example, adhesive, chemical, and electromagnetic radiation such as microwave, infrared and radiant energy.

Still another method of making a nonwoven fabric of the present invention is to combine meltblown fibers with spunbond continuous polymeric filaments. The meltblown fibers can contribute to bonding the spunbond filaments in two ways. According to one way, the spunbond filaments can be bonded after the web is separated from the forming surface. In such an embodiment, the spunbond filaments must be formed into a web which has sufficient integrity without adhesive bonding to be separated from the forming surface and then bonded without the surface features of the fabric disintegrating. This be accomplished by combining meltblown polymeric fibers with the spunbond filaments to form the web whereby the spunbond filaments and the meltblown fibers are entangled sufficiently so that the surface features of the web that are imparted by the forming surface remain intact during the separating and bonding steps. According to yet another method way of bonding with meltblown fibers, the spunbond filaments can be bonded before or after the separation step. According to this method, adhesive meltblown fibers are combined with the spunbond continuous filaments and the resulting web is heated to activate the adhesive fibers.

Meltblown processes of making nonwoven fabrics are known. Suitable meltblowing techniques and SMS fabrics are disclosed in U.S. Pat. No. 4,041,203, the disclosure of which is incorporated herein by reference. U.S. Pat. No. 4,041,203 references the following publications on meltblowing techniques which are also incorporated herein by reference: an article entitled “Superfine Thermoplastic Fibers” appearing in Industrial Engineering Chemistry, Volume 48, Number 8, ppgs. 1342-1346 which describes work done at the U.S. Naval Research Laboratories in Washington, D.C.; Naval Research Laboratory Report 111437, dated Apr. 15, 1954; U.S. Pat. Nos. 3,715,251; 3,704,198; 3,676,242; and 3,595,245; and British Specification No. 1,217,892.

Spunbond meltblown integrated composite (SMIC) materials are described and illustrated in the previously mentioned U.S. Pat. Nos. 5,575,874 and 5,643,653 issued to Griesbach et al. Generally, an SMIC material can be produced by meltblowing material on each side of a spunbond filament curtain. Meltblowing dies can be positioned on each side of the spunbond filament curtain in a symmetric fashion to produce a SMIC fabric. The process is described in more detail in U.S. Pat. Nos. 5,575,874 and 5,643,653 issued to Griesbach et al. which are incorporated by reference herein. The SMIC fabric that is formed can be positioned on a surface that includes topographical features and bonded with hot air to provide a SMIC fabric that includes surface features.

The surface including topographical features may take on many configurations in the practice of the present invention. Generally described the surface including topographical features used with the present invention is shaped and desirably includes an array of discrete topographical features. Generally the topographical features are projections but may be recesses or include both recesses and projections. A nonwoven web formed on a surface including topographical features as described herein conforms to a shape that corresponds to the shape of the forming surface. The resulting nonwoven fabric may include projections and/or indentations and even apertures.

The surface features of the forming surfaces used in the methods of the present invention each have a cross-section width (W) extending between adjacent land areas.

The cross-sections width (W) of at least some of the individual surface features of the forming surface have a minimum dimension of at least about ⅛ of an inch, 5/32 of an inch, or at least about 3/16 of an inch and even up to and exceeding ¼ of an inch. Thus, when a nonwoven fabric is formed on such a surface the resulting fabric also has topographical features with approximately the same corresponding cross-sectional width (W). For example, when the surface features of the forming surface include projections recesses, the forming surface has a length and a width which define a reference surface area and the recesses or projections each have an open cross-sectional area which forms part of the reference surface area and extend between adjacent land areas. The open cross-sectional areas of the recesses desirably total from about 10 percent to about 95 percent of the reference surface area and more desirably from about 25 percent to about 50 percent of the reference surface area. The recesses desirably have a depth of at least about ⅛ of an inch, 5/32 of an inch, 3/16 of an inch and even exceeding ¼ of an inch. The cross-section of the recesses extending between the adjacent land areas more desirably has a minimum dimension of at least about 3/32 of an inch, more desirably at least about ⅛ of an inch, 5/32 of an inch, 3/16 of an inch and even exceeding ¼ of an inch.

An example of a forming surface in accordance with an exemplary embodiment of the present invention is illustrated in FIG. 2. FIG. 2 schematically illustrates the materials that were used as the forming surfaces in Examples 1 and 2. The materials were purchased from SpaceNet Inc. of Monroe, N.C. and are marketed as a cushioning material under the trademark SPACENET. The SPACENET materials are synthetic thermoplastic fiber networks having topographical features as illustrated and described in U.S. Pat. Nos. 5,731,062, 5,851,930 and 6,007,898. Generally, SPACENET material is a woven network of polyester fibers 200 as illustrated in FIG. 2 that is thermoformed into a pattern having topographical features 220 of approximately ⅜ of an inch in diameter and ¼ inch in height. The topographical features 220 of this cushioning material are arranged in a repeating pattern as illustrated in FIG. 2 and are spaced apart from each other by land areas 210 that are approximately ¼ of an inch in width. The SPACENET material provided a forming surface that was highly air permeable. It would be desirable to provide a forming surface including such features that is strong enough and durable enough to handle the requirements of repeated use and that eliminates the need for a separate carrying wire. The SPACENET material 300 includes a plurality of “hat-shaped” projections 220 on a base area 210. The SPACENET network is formed from polyester fibers that are woven and thermoformed in a pattern having macroscopic surface features of approximately ¼ inch in height.

When used as a forming wire, the SPACENET material provides a nonwoven web with a unique pattern and geometry that corresponds to the SPACENET pattern and macroscopic surface features. The SPACENET material has uniform permeability throughout to evenly allow air flow through the nonwoven and subsequently through the wire below. The sizes, heights, shapes and spacings of the pattern of projections 220 of the SPACENET material and the forming and bonding wires can vary. Thus, surface features 120 of web 100 the can vary with the surface features 220 of the forming wire 200. Any number of patterned surface wires may be used in the present invention as long as the wire provides the web with surface features. Although the surface 200 having surface features 220 is illustrated as a surface having features uniformly distributed in both the machine direction (MD) and the cross direction (CD), the features can be uniformly distributed in only one direction, either the machine direction or the cross direction. Furthermore, the features 220 do not necessarily have to be uniformly provided or distributed on the surface 200 and can be provided and distributed in any pattern.

In the exemplary embodiment, the SPACENET material 200 was situated on and supported by a conventional carrying wire 75 during the web making process. The SPACENET material used as a forming surface may also be used as a topographical bonder wire and/or a forming wire, upon which a nonwoven web is shaped to provide the nonwoven web 100 with a unique pattern and geometry of macroscopic features 120 separated by land areas 110 that corresponded to the pattern and geometry of the macroscopic topographical features of the SPACENET material. The SPACENET material advantageously has uniform permeability throughout and allows uniform and high air flow through the nonwoven web, the forming surface and the carrying wire 75 below. The high, uniform permeability of the SPACENET material is particularly desirable for nonwoven forming processes. SPACENET cushioning material is available in different sizes and patterns under the material designations. SPACENET cushioning materials are sold with a variety of surface feature sizes and patterns, for example under the designations K15003, K15005 and K30008.

Desirably, the surface and surface features has a uniform open area that desirably has an percentage of open area that is greater than 10 percent, more desirably having more than 15 percent open area and even more than 20 percent open area. The surface and surface features should have uniform permeability, desirably greater than 300 cubic feet per minute (cfm) and more desirably 500 cfm and greater. It would also be desirable to provide a surface including such features that is strong enough and durable enough to handle the requirements of repeated use and that eliminates the need for a separate carrying wire during the web making process. A metal or plastic wire or surface having surface features may be used to provide such a shape-inducing surface

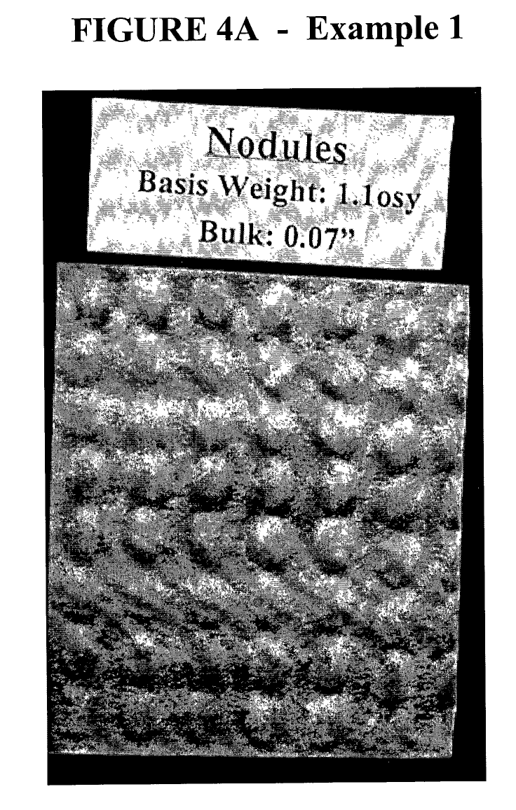

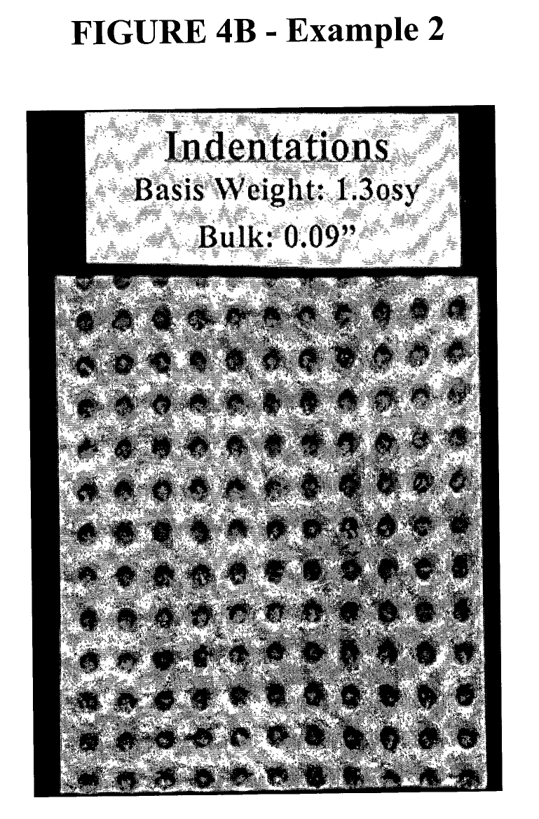

Photographs of two nonwoven fabrics made on a SPACENET surface are provided in FIGS. 4A and 4B. The fabric illustrated in FIG. 4A was formed on SPACENET K15005 cushioning material with the projections facing downward. The fabric illustrated in FIG. 4B was formed on SPACENET K15003 cushioning material with the projections facing upward. SPACENET cushioning materials are available in many patterns and sizes other than the pattern and size illustrated in FIG. 2 or used in the examples. The SPACENET material may be oriented with the projections facing upward or downward to provide different forming surfaces and different nonwovens formed on the forming surfaces.

Generally, the forming surface may include any number, size and/or pattern of surface features. The surface features may include projections and/or recesses.

However, it is desirable that the surface features are foraminous and permeable to gas. More desirably, the surface features have permeability that is similar to the rest of the forming surface so that air used to form and/or bond the fabric web on top of the forming surface can permeate the surface features and the forming surface to deposit fibers on the surface and the features in a uniform manner. Generally, the surface features 220 of the forming surface 200 are separated by land areas 210 as illustrated in FIG. 2. The surface features 220 have a cross-section width (W), which extends between adjacent land areas 210 and form part of the reference surface area. The surface features 220 have a minimum dimension, for example, a height (H), a depth, a length or a width (W), of at least about ⅛ of an inch, 5/32 of an inch, 3/16 of an inch and even exceeding ¼ of an inch.

To form a fabric adapting conventional methods of making nonwoven fabrics, the land areas 210 of the forming surface 200 and the surface features 220 are foraminous and are permeable to gas. More desirably, the land areas 210 and the surface features 220 are relatively equal in permeability to air. As a result, when fibers are collected on or a fabric is formed on top of the forming surface 200, the continuous filaments are drawn by the vacuum beneath the forming surface substantially uniformly into any recesses, over any projections or other surface features as well as the land areas because the pressure drop across the features is substantially the same as the pressure drop across the land areas or the bulk of the forming surface. Thus, the resulting fabric has a shape which corresponds to the shape of the forming surface 200 and the fabric projections are substantially filled with filaments. Like the forming surface 200, the fabric 100 produced thereon includes land areas 110 and surface features 120. In at least one embodiment, the land areas 110 of the fabric 100 correspond to the land areas 210 of the forming surface 200 and projections in the fabric correspond to projections or recesses in the forming surface.

A fabric 100 may also include features formed in recesses in the forming surface 200. The surface features of the nonwoven fabrics of the present invention are separated by land areas 210 of the fabric. Nonwoven fabrics produced by the methods of the present invention can be used components in personal care articles, sound proofing materials and so forth. Like the forming surfaces 200, the fabrics 100 of the invention may include projections 120 or depressions that have a minimal dimension W which is at least about ⅛ of an inch, 5/32 of an inch, 3/16 of an inch and even exceeding ¼ of an inch. Like the forming surface 200, the cross-sectional areas of the projections 120 of the fabric 100 desirably total from about 10 percent to about 95 percent of the referenced surface area of the fabric, and more desirably from about 20 percent to about 50 percent of the reference surface area of the fabric thus decreasing the contact area of a body with a fabric of the invention.

Although the forming surfaces described above include a synthetic thermoplastic fiber network having topographical features attached to a mesh support wire, there are other methods of making such forming surfaces. In addition, the forming surface can be made by thermoforming a plastic wire mesh such as a polyester wire mesh into a configuration wherein the mesh has an array of projections separated by land areas. When it is desired to make an apertured nonwoven fabric, the surface features of the forming surface can also include nonporous projections as well as porous projections separated by foraminous areas. For example, every other projection may be non-porous to provide apertures at every other surface feature.

A separation layer or body side liner material can be made with a forming surface having a relatively small number of widely spaced projections. In one particular embodiment, the present invention includes a nonwoven structure having macroscopic surface features that can be used to separate one surface from another surface, for example, a baby's bottom from an absorbent layer of a diaper. In several desirable embodiments, the structure has physical, aesthetic, and functional attributes that are particularly desirable for use as a body-side liner; a surge material or a liner/surge combination in disposable absorbent products such as: diapers; training pants; incontinent pads; feminine hygiene products such as feminine pads, sanitary napkins, and pantiliners; and so forth.

In one desirable embodiment, a composite material including a three-dimensional surface of a woven network of polyester fibers including a pattern of macroscopic features and a nonwoven fabric formed on the woven network is provided. This composite material may be used as a soundproofing or insulating material. While this is contemplated as being one of many desirable uses, it should be understood that the present invention also has utility in a wide variety of absorptive devices, both disposable and reusable, such as sanitary napkins, catamenial tampons, incontinent pads, and so forth and in non-absorptive devices, such as industrial materials, sound proofing, insulation, packaging and so forth. The detailed description of the top-sheet structure and its use in a disposable diaper will allow those skilled in the art to readily adapt the invention to other devices.

Although a spunbond method of making a nonwoven fabric including surface features is described with reference to FIG. 1, structures having macroscopic surface features, including nonwoven fabrics and composites including nonwoven fabrics, can be made by a variety of methods. For example, macroscopic surface features can be imparted to an already formed web or a web can be formed with surface features. In FIG. 1, the nonwoven fabric web 100 is formed from continuously spun filaments 10 deposited on a wire 200. Methods of making spunbonded webs are known. Methods of making spunbonded webs are described in U.S. Pat. No. 3,802,817 issued to Matsuki et al. and U.S. Pat. Nos. 5,575,874 and 5,643,653 issued to Griesbach et al. which are herein incorporated by reference in their entirety. Synthetic polymer is extruded into filaments through extruders 20 and spin pack 30. The filaments are drawn through quench zone 40 and fiber draw unit 50. As a result, the diameters of the filaments are reduced and continuous filaments 10 are formed. The continuous filaments are deposited on a wire 60 to form a nonwoven synthetic fiber web. The nonwoven web is then exposed to a hot air knife 70 to provide the web with sufficient integrity to be transferred to second flat bonder wire (not shown) upon which is situated a forming 200 wire including surface features, e.g. SPACENET material. Alternatively, the forming wire 200 that imparts the surface features may extend through the whole process. That is, the forming wire 200 could be used as both the forming wire and a bonding wire or a bonder wire alone. The web 100, bonder wire and forming wire proceed to Through Air Bonder 80 where the web is exposed to hot air conforming the web 100 to the forming wire 200 providing the web 100 with surface features corresponding to the surface features 120 possessed by the forming wire 200 and providing the web 100 with additional integrity. The examples include bicomponent spunbond webs formed on top of a SPACENET material.

Those of skill in the art will appreciate that a web of the present invention can be made via other methods of making webs other than spunbond methods, for example meltblown and airlaid methods of making nonwovens. Additionally, the nonwoven web may be a bonded-carded web (BCW), a coform web, an airlaid web, a spunbond/meltblown/spunbond (SMS) web and so forth. Additionally, the webs can be formed from or include a variety of materials, cellulose, pulp fibers, bicomponent fibers, and so forth. Furthermore, the processes are not limited to one bank processes. A two-bank process may be used to provide for fiber gradients to be created. Large fibers can be produced in a first bank and small fibers in a second bank. In addition, the fibers in one or both banks can be treated with a wettable surfactant to produce a hydrophobicity gradient. Other modifications and treatments known to those of skill in the art may be used with the present invention.

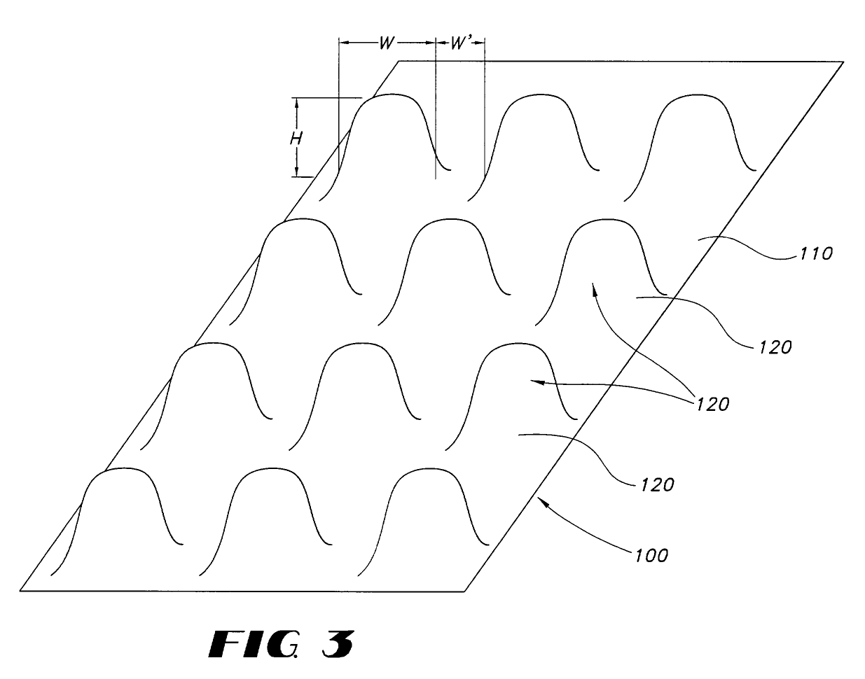

Web 100 is formed to possess a plurality of macroscopic surface features 120 and can be removed from the forming wire 200 and collected on winder 90 for later use and incorporation into other products. Alternatively, the forming wire 200 is not separated from the web 100 to provide a composite nonwoven/woven material that can be used for various purposes, e.g. sound proofing material. A web produced by the method illustrated in FIG. 1 and described in greater detail in Example 1 is shown in schematically illustrated in FIG. 3. The web 100 includes a plurality of macroscopic features, in this example discrete projections 120 that extend from the top surface 110 of the web. The projections 120 have distal surfaces 122. The distal surfaces 122 provide separation and define a top plane that is the plane that a substantially planar article would rest on if an article was resting on the surface features 120 and their respective distal surfaces 122. The top plane of the web is separated from a basal plane 110 of the web. The basal plane 110 is defined by the substantially planar portion of the top surface 110 of the non-raised areas below the surface features and the top plane.

Webs of the present invention may be treated with optional treatments and/or additives. For example, portions of the webs may be treated with a hydrophilic or a hydrophobic treatment to increase or decrease fluid intake of the treated portions. If the non-raised areas of the web are treated with a hydrophilic additive, the resulting non-treated raised areas, i.e. the surface features, will be more hydrophobic relative to the treated non-raised areas.

### EXAMPLE 1

In this Example 1, a nonwoven synthetic fabric web 100 having macroscopic surface features 120 was prepared on a forming surface 200 including similar macroscopic surface features 220 according to the process generally illustrated in FIG. 1 and described below. The materials that were used as the forming surfaces in this Example 1 and Example 2 purchased from SpaceNet Inc. of Monroe, N.C. and is marketed as a cushioning material under the trademark SPACENET. The SPACENET material is a synthetic thermoplastic fiber network having surface features as illustrated and described in U.S. Pat. Nos. 5,731,062, 5,851,930 and 6,007,898. The SPACENET material in this example was SPACENET K15005. Generally, the SPACENET K15005 material is a woven network of polyester fibers 200 as illustrated in FIG. 2 that is thermoformed into a pattern having surface features 220 of approximately ⅜ of an inch in diameter and ¼ inch in height. The surface features 220 of this cushioning material are arranged in a repeating pattern as illustrated in FIG. 2 and are spaced apart from each other by land areas 210 that are approximately ¼ of an inch in width. The SPACENET material provided a forming surface that was highly air permeable. The SPACENET material 200 was situated on and supported by a conventional carrying wire 75 with the projections facing downward during the web making process, specifically the TAB.

The nonwoven fabric web 100 of this Example 1 and the following Example 2 was formed from continuous bicomponent filaments 10 under the conditions described below. The bicomponent filaments 10 were made from approximately equal amounts of two polymer components in a side-by-side configuration. The composition of the first component was 98% by weight of 3445 polypropylene from Exxon of Houston, Tex. and 2% by weight of titanium dioxide. The composition of second component was 100% by weight of XUS 61800.41 polyethylene from Dow Chemical Company of Midland, Mich. The spin hole geometry of the spin pack 30 was 0.6 mm diameter with a length to diameter (L/D) ratio of 4:1 and the spinneret had 50 holes per inch in the cross direction. The melt temperature in the spin pack was 410° F. and the throughput was 0.6 grams/hole/minute (ghm). The forming height was 12 inches. The quench air flow rate of the air quencher 40 was 32 standard cubic feet per minute (scfm) and the temperature was 50° F. The aspirator temperature was ambient, approximately 75° F., and the aspirator pressure was 3.5 pounds per square inch (psi). The hot air knife (HAK) 70 was at 270° F. inlet air with an exit air temperature of 180° F., the pressure was 0.8 psi and the height of the HAK above the wire was 1.5 inches. The under wire vacuum 65 air was at 7 inches water. The line speed was adjusted to produce a nonwoven web with a basis weight of 1.1 ounces per square yard (osy). The unbonded nonwoven 100 was transferred onto a forming surface 200 supported by carrying wire 75 which proceeded to a Through Air Bonder (TAB) 80 as illustrated in FIG. 1. The SPACENET forming surface 300 was situated on and attached to the carrying wire 75 which was a regular flat bonder wire. The forming surface 200 and nonwoven web 100 were placed in the TAB 80 which was set at an air temperature of approximately 280° F. and 0.6 psi of air pressure and exhaust to form and bond the fibers into an integrated web with macropscopic surface features. A photograph of the web of Example 1 is provided in FIG. 4A.

SPACENET K15005 cushioning material with the projections facing up was used as a surface having macroscopic features, this shape-inducing surface may be used as a surface bonder wire and/or a forming wire, upon which a nonwoven web is shaped provided the nonwoven web 100 with a unique pattern and geometry of macroscopic features 120 separated by land areas 110 that corresponded to the pattern and geometry of the macroscopic surface features of the SPACENET material. The SPACENET material advantageously has uniform permeability throughout and allows uniform and high air flow through the nonwoven web, the forming surface and the carrying wire 75 below. The high, uniform permeability of the SPACENET material is particularly desirable for nonwoven forming processes.

### EXAMPLE 2

A nonwoven web was produced in a similar process as described in above Example 1 with the exception that the SPACENET material was SPACENET K15003 having smaller bump and the line speed was adjusted to produce a nonwoven web with a basis weight of 1.3 ounces per square yard (osy). Additionally, the SPACENET K15003 material was extended and served as both a forming surface and a bonding surface and was placed on a support wire with the projections facing upward. Specifically, a continuous forming and bonding surface made of SPACENET material supported by carrying wires 60 and 75 was used throughout the web making process from the forming of the filaments into a web under the fiber draw unit 50 and the transporting the web through the TAB 80 where the web was formed and bonded into an integral web having macroscopic surface features. The nonwoven synthetic web of this Example 2 was formed onto the SPACENET material under the same conditions as described above with the exception that that the SPACENET material was used as a forming surface and as well as a bonding surface. A photograph of the nonwoven fabric that was produced by this second example is provided in FIG. 4B.

While the invention has been described in detail with respect to specific embodiments thereof, it will be appreciated that those skilled in the art, upon attaining an understanding of the foregoing may readily conceive of alterations to, variations of and equivalents to these embodiments. Accordingly, the scope of the present invention should be assessed as that of the appended claims and any equivalents thereto.

We claim:

1. A process of making a nonwoven fabric comprising
providing a three-dimensional surface that comprises surface features that are air permeable,
depositing fibers or a web comprising fibers onto the surface, and
stabilizing the fibers to form a nonwoven fabric.

2. The process of claim 1, wherein the surface features have an air permeability that is substantially equal to the air permeability of the portion or portions of the surface that does not comprise surface features.

3. The process of claim 1, wherein the surface features comprise foraminous areas that are air permeable.

4. The process of claim 1, wherein the surface comprises a plurality of surface features at least one of the surface features is greater than ⅛ of an inch in height

5. The process of claim 1, wherein the surface comprises a plurality of surface features at least one of the surface features is greater than 5/32 of an inch in height.

6. The process of claim 1, wherein the surface comprises a plurality of surface features that are macroscopic and at least one of the surface features is greater than a 3/16 of an inch in height.

7. The process of claim 1, wherein the surface comprises a permeable metal surface.

8. The process of claim 1, wherein the surface is substantially uniformly permeable over the majority of the surface.

9. The process of claim 1, wherein the fibers are deposited by a process selected from meltspun, spunbond, meltblown, coform, airlaid and bonded carded web processes.

10. The process of claim 1, further comprising depositing the fibers on a first surface and then transferring the fibers to the three-dimensional surface that comprises surface features that are air permeable.

11. The process of claim 1, further comprising bonding the fibers at elevated temperature.

12. A process for making a nonwoven fabric comprising the steps of:
a. providing polymeric filaments;
b. depositing the polymeric filaments on a surface that comprises surface features, wherein the surface and the surface features are air permeable;
c. forcing air or another gas or liquid through the polymeric filaments, the surface and the surface features to arrange the polymeric filaments into a web; and
d. bonding the polymeric filaments to integrate the web.

13. The process of claim 12, wherein the surface features comprise foraminous areas that are air permeable.

14. The process of claim 13, wherein a plurality of the surface features have a height that is greater than ⅛ of an inch.

15. The process of claim 12, wherein a plurality of the surface features are macroscopic and have a height that is greater than 3/16 of an inch.

16. The process of claim 12, wherein a plurality of the surface features are macroscopic and have a height that is greater than ¼ of an inch.

17. The process of claim 12, wherein the step of providing the polymeric filaments comprises forming polymeric filaments.

18. The process of claim 17, wherein forming the polymeric filaments comprises melt spinning polymeric filaments.

19. The method of claim 18, further comprising drawing the polymeric filaments prior to forming on a surface.

20. The method of claim 19, further comprising quenching the filaments prior to drawing the filaments.

21. The method of claim 12, further comprising separating the web from the surface.

22. The process of claim 18, wherein melt spinning comprises a method selected from the group consisting of meltspun, spunbond, meltblown, coform, air laid and bonded carded web processes.

23. The process of claim 12, further comprising providing cellulose-based fibers depositing the cellulose-based filaments on the surface that comprises surface features.

24. The process of claim 12, wherein forcing air or another gas or liquid through the polymeric filaments, the surface and the surface features to arrange the polymeric filaments into a web comprises forcing air at elevated temperature and pressure through the polymeric filaments, the surface and the surface features.

25. The process of claim 12, further comprising depositing the polymeric fibers on a first surface and then depositing the polymeric filaments on the surface that comprises surface features, wherein the surface and the surface features are air permeable.

26. A nonwoven fabric made by the method of claim 1.

27. A personal care product comprising as a component the nonwoven fabric of claim 26.

28. A composite material comprising the nonwoven fabric of claim 26 and the three-dimensional surface, wherein the three-dimensional surface is a woven network of polyester fibers including a pattern of macroscopic features.

29. A nonwoven fabric made by the method of claim 12.

30. A personal care product comprising as a component the nonwoven fabric of claim 29.
